(19) United States
(12) Patent Application Publication
(10) Pub. No.: US 2024/0417428 A1
(43) Pub. Date: Dec. 19, 2024

(54) POLYPEPTIDE FOR INHIBITING TRPM8 AND USE THEREOF

(71) Applicant: ZHEJIANG UNIVERSITY, Hangzhou City, Zhejiang Province (CN)

(72) Inventors: Fan YANG, Hangzhou City, Zhejiang Province (CN); Aierken AERZIGULI, Hangzhou City, Zhejiang Province (CN)

(21) Appl. No.: 18/701,434

(22) PCT Filed: Oct. 14, 2022
(86) PCT No.: PCT/CN2022/125397

§ 371 Date: Apr. 15, 2024

(30) Foreign Application Priority Data
Oct. 14, 2021 (CN) 202111197510.9

(51) Int. Cl. C07K 7/08; A61K 38/00; A61P 25/02; A61P 25/04

(52) U.S. Cl. CPC C07K 7/08; A61P 25/02; A61P 25/04; A61K 38/00

(57) ABSTRACT

The present invention relates to a polypeptide for inhibiting TRPM8 and a use thereof. Specifically, the present invention provides a polypeptide or a pharmaceutically acceptable salt thereof. The polypeptide of the present invention has an excellent inhibitory effect on TRPM8 and is used for preventing and treating TRPM8-related diseases.

### CROSS-REFERENCE TO RELATED APPLICATION

This patent application claims priority to the Chinese patent application No. 2021111975109 filed on Oct. 14, 2021, and titled “A polypeptide for inhibiting TRPM8 and a use thereof”, which is incorporated herein by reference in its entirety.

### TECHNICAL FIELD

The present invention relates to the field of medicine, specifically to a polypeptide for inhibiting TRPM8 and a use thereof.

### BACKGROUND

Peripheral neuropathy is a common side effect of many platinum-based chemotherapy regimens, which limits the dosage of anticancer drugs and reduces the quality of life of patients taking these drugs. For example, oxaliplatin is commonly used as first-line chemotherapy in the treatment of many tumors, such as colorectal cancer and gastric cancer. However, up to 89% of patients taking oxaliplatin will develop acute neurotoxicity, which usually manifests as extreme coldness and excruciating pain in the arms and legs in normal cool ambient temperatures. Although symptoms of these neuropathies, including oxaliplatin-induced cold allodynia, improve in patients after stopping taking the drug, there is still no effective treatment for this type of neuropathy to date.

TRPM8 is a type of non-selective ion channel expressed on nociceptive neurons. This channel is activated at temperatures below 28° C., making it a mammalian cold receptor. In addition, as a multimodal receptor, TRPM8 can also be activated by a variety of physical stimuli or chemical ligands, such as menthol and Icilin, as well as transmembrane depolarization. In a mouse model of oxaliplatin-induced cold allodynia, the expression levels of TRPM8 channels were significantly increased in nociceptive dorsal root ganglion (DRG) neurons. More importantly, administration of oxaliplatin did not cause cold allodynia in TRPM8 knockout mice. Therefore, TRPM8 ion channel is an effective drug target for peripheral neuropathy and hyperalgesia caused by platinum drugs. There is an urgent need to develop effective drugs targeting TRPM8 ion channel for the treatment of peripheral neuropathy and hyperalgesia.

### SUMMARY OF THE INVENTION

The object of the present invention is to provide a TRPM8 inhibitor and a polypeptide for preventing and treating TRPM8-related diseases.

In a first aspect of the present invention, a polypeptide or a pharmaceutically acceptable salt thereof is provided, which has a structure represented by Formula I:

X1-X2-X3-X4-X5-X6-X7-X8-X9-X10-X11-X12-X13 Formula I

In the Formula,
X1 is none, any amino acid or any peptide fragment;
X2 is R, K, Q, A, D or N;
X3 is R, K, Q, A, H or N;
X4 is D, A, S, R or E;
X5 is R, K, Q, A or N;
X6 is A, V, L, G, R or I;
X7 is R, K, Q, A or N;
X8 is H, N, Q, K, A or R;
X9 is Y, W, F, D, T, A, R or S;
X10 is R, K, Q, A, S, Y or N;
X11 is Q, A, R or N;
X12 is R, K, Q, A, K or N;
X13 is none, any amino acid or any peptide fragment.

In another preferred embodiment, X1, X2, X3, X4, X5, X6, X7,

In another preferred embodiment, X2 is R, K, Q, D or N.

In another preferred embodiment, X6 is A, V, L, R or I.

In another preferred embodiment, X12 is R, K, Q, K or N.

In another preferred embodiment, the polypeptide or the pharmaceutically acceptable salt thereof has uses of: (a) inhibiting TRPM8; (b) preventing and/or treating TRPM8-related diseases; (c) preventing and/or treating cold pain sensation Hypersensitivity; (d) preventing and/or treating peripheral neuropathy; (e) preventing and/or treating pain; and/or (f) preventing and/or treating chronic constriction injury of neuropathic pain.

In another preferred embodiment, the polypeptide is an isolated polypeptide.

In another preferred embodiment, the polypeptide is artificially synthesized.

In another preferred embodiment, the polypeptide is a recombinant polypeptide.

In another preferred embodiment, X1 is None, C (cysteine) or M (methionine).

In another preferred embodiment, X13 is None or C (cysteine).

In another preferred embodiment, the peptide fragment includes a tag protein, a leader sequence or a secretion sequence.

In another preferred embodiment, the length of the X1 is 1-20 aa, more preferably 1-10 aa, most preferably 1-5 aa.

In another preferred embodiment, the length of the X13 is 1-20 aa, more preferably 1-10 aa, most preferably 1-5 aa.

In another preferred embodiment, the length of the polypeptide or the pharmaceutically acceptable salt thereof is ≤25 aa, preferably ≤20 aa, more preferably ≤18 aa, even more preferably ≤15 aa; most preferably 13 aa, 14 aa, 15 aa, 16 aa, 17 aa, 18 aa, 19 aa or 20 aa.

In another preferred embodiment, the X1 or X13 includes natural or unnatural amino acids.

In another preferred embodiment, a cyclic peptide is formed between the X1 and the X13.

In another preferred embodiment, at least one pair of disulfide bonds are formed between the X1 and the X13.

In another preferred embodiment, a pair of disulfide bonds is formed between the X1 and the X13.

In another preferred embodiment, the polypeptide has the structure shown in Formula II:

X1-RRDRARHYRQR-X13 Formula II
wherein, X1 and X13 are defined as above.

In another preferred embodiment, the polypeptide is an N-polymer.

In another preferred Formula, the N-polymer has the structure shown in Formula III:

—(X1-RRDRARHYRQR-X13-L1)n- Formula III
wherein, X1, X13 are defined as above; L1 is none or a linked peptide; n is 2˜10, preferably 2˜7, more preferably 2˜5; each “-” is independently a linked peptide or a peptide bond.

In another preferred embodiment, the L1 is none.

In another preferred embodiment, the length of the L1 is 1-30 aa, preferably 1-20 aa, more preferably 1-10 aa.

In another preferred embodiment, n is 2, 3 or 4.

In another preferred embodiment, each “-” is independently a peptide bond.

In another preferred embodiment, a sequence of the polypeptide are as follows: SEQ ID NO: 1, SEQ ID NO:2, SEQ ID NO:3, SEQ ID NO:4, SEQ ID NO:5, SEQ ID NO: 6, SEQ ID NO:7, SEQ ID NO:8, SEQ ID NO:9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 19.

In one preferred embodiment, compared with the polypeptide shown in SEQ ID NO: 1, SEQ ID NO:2, SEQ ID NO:3, SEQ ID NO:4, SEQ ID NO:5, SEQ ID NO: 6, SEQ ID NO:7, SEQ ID NO:8, SEQ ID NO:9, SEQ ID NO: 10, SEQ ID NO: 11, SEQ ID NO:12, SEQ ID NO:13, SEQ ID NO: 14, SEQ ID NO: 15, SEQ ID NO: 16, SEQ ID NO: 17, SEQ ID NO: 18 or SEQ ID NO: 19, the polypeptide shown in the Formula I has ≥50%, ≥60%, ≥70%, ≥80%, ≥90%, ≥95%, ≥99% or 100% homology (or homogeny).

In one preferred embodiment, the polypeptide shown in the Formula I retains at least ≥50%, ≥60%, ≥70%, ≥80%, ≥90%, ≥95%, or ≥99%, e.g. 90-100% of the biological activity of the polypeptide shown in SEQ ID NO:1, SEQ ID NO:2, SEQ ID NO:3, SEQ ID NO:4, SEQ ID NO:5, SEQ ID NO: 6, SEQ ID NO: 7, SEQ ID NO:8, SEQ ID NO:9, SEQ ID NO: 10, SEQ ID NO:11, SEQ ID NO: 12, SEQ ID NO: 13, SEQ ID NO:14, SEQ ID NO: 15, SEQ ID NO: 16, SEQ ID NO: 17, SEQ ID NO: 18 or SEQ ID NO: 19.

In another preferred embodiment, the sequence of the polypeptide is shown in SEQ ID NO:1.

In another preferred embodiment, compared with the polypeptide shown in SEQ ID NO: 1, the polypeptide shown in the Formula I has ≥50%, ≥60%, ≥70%, ≥80%, ≥90%, ≥95%, ≥99% or 100% homology (or homogeny).

In another preferred embodiment, the polypeptide shown in the Formula I retains at least ≥50%, ≥60%, ≥70%, ≥80%, ≥90%, ≥95%, ≥99%, for example 90-100% of the biological activity of the polypeptide shown in SEQ ID NO: 1.

In another preferred embodiment, the polypeptide is selected from the following group:
(1) a polypeptide that has the amino acid sequences shown in SEQ ID NO:1, SEQ ID NO:2, SEQ ID NO:3, SEQ ID NO:4, SEQ ID NO:5, SEQ ID NO:6, SEQ ID NO: 7, SEQ ID NO: 8, SEQ ID NO:9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 19;
(2) a polypeptide that is formed by substitution, deletion or addition of 1-5 (preferably 1-3, more preferably 1-2) amino acid residues in the amino acid sequence shown in SEQ ID NO:1, SEQ ID NO:2, SEQ ID NO:3, SEQ ID NO:4, SEQ ID NO:5, SEQ ID NO:6, SEQ ID NO:7, SEQ ID NO: 8. SEQ ID NO:9, SEQ ID NO: 10, SEQ ID NO:11, SEQ ID NO: 12, SEQ ID NO:13, SEQ ID NO:14, SEQ ID NO: 15, SEQ ID NO:16, SEQ ID NO:17, SEQ ID NO:18 or SEQ ID NO: 19 and has the uses of: (a) inhibiting TRPM8; (b) preventing and/or treating TRPM8-related diseases; (c) preventing and/or treating cold allodynia; (d) preventing and/or treating peripheral nerves lesions; (e) preventing and/or treating pain; and/or (f) preventing and/or treating chronic constriction injury of neuropathic pain;
(3) a polypeptide that has ≥50%, ≥60%, ≥70%, ≥80%, ≥90%, ≥95%, ≥99% or 100% homology (or homogeny) compared with a polypeptide shown in SEQ ID NO:1, SEQ ID NO:2, SEQ ID NO:3, SEQ ID NO:4, SEQ ID NO:5, SEQ ID NO: 6, SEQ ID NO: 7, SEQ ID NO: 8, SEQ ID NO:9, SEQ ID NO: 10, SEQ ID NO: 11, SEQ ID NO:12, SEQ ID NO:13, SEQ ID NO:14, SEQ ID NO: 15, SEQ ID NO: 16, SEQ ID NO: 17, SEQ ID NO: 18 or SEQ ID NO: 19.

In another preferred embodiment, the polypeptide is not SEQ ID NO:3.

In another preferred embodiment, the polypeptide is selected from the following groups:
(1) a polypeptide that has the amino acid sequences shown in SEQ ID NO:1, SEQ ID NO:2, SEQ ID NO:5, SEQ ID NO:6, SEQ ID NO:7, SEQ ID NO:9, SEQ ID NO: 10, SEQ ID NO: 11, SEQ ID NO:12, SEQ ID NO:13, SEQ ID NO:15, SEQ ID NO:16, SEQ ID NO:17, SEQ ID NO:18 or SEQ ID NO:19;
(2) a polypeptide that is formed by substitution, deletion or addition of 1-5 (preferably 1-3, more preferably 1-2) amino acid residues in the amino acid sequence shown in SEQ ID NO:1, SEQ ID NO:2, SEQ ID NO:5, SEQ ID NO:6, SEQ ID NO:7, SEQ ID NO:9, SEQ ID NO: 10, SEQ ID NO: 11, SEQ ID NO: 12, SEQ ID NO: 13, SEQ ID NO:15, SEQ ID NO: 16, SEQ ID NO: 17, SEQ ID NO:18 or SEQ ID NO: 19 and has uses of: (a) inhibiting TRPM8; (b) preventing and/or treating TRPM8-related diseases; (c) preventing and/or treating cold allodynia; (d) preventing and/or treating peripheral neuropathy; (e) preventing and/or treating pain; and/or (f) preventing and/or treating chronic constriction injury of neuropathic pain;
(3) a polypeptide that has ≥50%, ≥60%, ≥70%, ≥80%, ≥90%, ≥95%, ≥99% or 100% homology (or homogeny) compared with a polypeptide shown in SEQ ID NO:1, SEQ ID NO:2, SEQ ID NO:5, SEQ ID NO:6, SEQ ID NO: 7, SEQ ID NO: 9, SEQ ID NO: 10, SEQ ID NO: 11, SEQ ID NO:12, SEQ ID NO: 13, SEQ ID NO: 15, SEQ ID NO: 16, SEQ ID NO: 17, SEQ ID NO: 18 or SEQ ID NO: 19.

In another preferred embodiment, the polypeptide is selected from the following group:
(1) a polypeptide having the amino acid sequence shown in SEQ ID NO: 1;
(2) a polypeptide that is formed by substitution, deletion or addition of 1-5 (preferably 1-3, more preferably 1-2) amino acid residues in the amino acid sequence shown in SEQ ID NO: 1 and has uses of: (a) inhibiting TRPM8; (b) preventing and/or treating TRPM8-related diseases; (c) preventing and/or treating cold allodynia; (d) preventing and/or treating peripheral neuropathy; (e) preventing and/or treating pain; and/or (f) preventing and/or treating chronic constriction injury of neuropathic pain;
(3) a polypeptide that has ≥50%, ≥60%, ≥70%, ≥80%, ≥90%, ≥95%, ≥99% or 100% homology (or homogeny) compared with a polypeptide shown in the polypeptide shown in SEQ ID NO.: 1.

In another preferred embodiment, the polypeptide is formed by substitution, deletion of 1-3, preferably 1-2, more preferably 1 amino acid; and/or addition of 1-5, preferably 1-4, more preferably 1-3, most preferably 1-2 amino acids in the polypeptide shown in SEQ ID NO: 1, SEQ ID NO: 2, SEQ ID NO: 3, SEQ ID NO: 4, SEQ ID NO: 5, SEQ ID NO: 6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO: 15, SEQ ID NO: 16, SEQ ID NO:17, SEQ ID NO: 18 or SEQ ID NO: 19.

In another preferred embodiment, the derivatized polypeptide retains ≥50%, ≥60%, ≥70%, ≥80%, ≥90%, ≥99%, or 100%, for example 80-100%, preferably 95-100% of (a) inhibiting TRPM8; (b) preventing and/or treating TRPM8-related diseases; (c) preventing and/or treating cold allodynia; and/or (d) to preventing and/or treating peripheral neuropathy of the polypeptide shown in SEQ ID NO:1, SEQ ID NO:2, SEQ ID NO:3, SEQ ID NO:4, SEQ ID NO:5, SEQ ID NO: 6, SEQ ID NO:7, SEQ ID NO:8, SEQ ID NO:9, SEQ ID NO: 10, SEQ ID NO: 11, SEQ ID NO: 12, SEQ ID NO: 13, SEQ ID NO: 14, SEQ ID NO: 15, SEQ ID NO: 16.

In another preferred embodiment, the derivatized polypeptide has ≥50%, preferably ≥60%, more preferably ≥70%, more preferably ≥80%, even more preferably ≥90%, most preferably ≥99% homology of SEQ ID NO:1, SEQ ID NO: 2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 19.

In another preferred embodiment, the polypeptide is formed by substitution, deletion of 1-3, preferably 1-2, more preferably 1 amino acid; and/or addition of 1-5, preferably 1-4, more preferably 1-3, most preferably 1-2 amino acids in the polypeptide shown in SEQ ID NO: 1.

In another preferred embodiment, the derivatized polypeptide retains ≥50%, ≥60%, ≥70%, ≥80%, ≥90%, ≥99%, or 100%, for example 80-100%, or preferably 95-100% of (a) inhibiting TRPM8; (b) preventing and/or treating TRPM8-related diseases; (c) preventing and/or treating cold allodynia; and/or (d) to preventing and/or treating peripheral neuropathy of the polypeptide shown in SEQ ID NO: 1.

In another preferred embodiment, the derived polypeptide has ≥50%, preferably ≥60%, more preferably ≥70%, more preferably ≥80%, even more preferably ≥90%, most preferably ≥99% homology of SEQ ID NO:1.

In a second aspect of the present invention, a fusion protein is provided, which includes:
(a) a polypeptide or a pharmaceutically acceptable salt thereof as described in the first aspect of the present invention;
(b) a peptide fragment fused to the polypeptide or a pharmaceutically acceptable salt thereof as described in the first aspect of the present invention.

In another preferred embodiment, the peptide fragment includes a vector protein.

In another preferred embodiment, the vector protein is selected from the following groups: Fc fragment, human serum albumin (HSA), CTP, transferrin, or a combination thereof.

In another preferred embodiment, the peptide fragment is undergone modification.

In another preferred embodiment, the modification includes polyethylene glycol (PEG) modification.

In a third aspect of the present invention, a polynucleotide is provided, which encodes a polypeptide or a pharmaceutically acceptable salt thereof as described in the first aspect of the present invention.

In another preferred embodiment, the polynucleotide is an isolated polynucleotide.

In a fourth aspect of the present invention, a vector is provided, which comprises the polynucleotide according to the third aspect of the present invention.

In another preferred embodiment, the vector includes a plasmid vector.

In a fifth aspect of the present invention, a host cell is provided, the host cell comprises the vector according to the fourth aspect of the present invention or the chromosome of the host cell integrates the polynucleotide according to the third aspect of the present invention.

In a sixth aspect of the present invention, a composition is provided, which comprises:
(a) a polypeptide or a pharmaceutically acceptable salt thereof according to the first aspect of the present invention; and
(b) a pharmaceutically acceptable vector or excipient.

In another preferred embodiment, the composition is a pharmaceutical composition.

In another preferred embodiment, the composition is administered by a method selected from the following group: intravenous, intratumoral, intracavity, subcutaneous or hepatic artery administration (such as injection, infusion, etc.).

In another preferred embodiment, the dosage form of the composition is an oral preparation, an injection preparation or an external preparation.

In another preferred embodiment, the dosage form of the composition is a solid preparation, a liquid preparation or a semi-solid preparation.

In another preferred embodiment, the preparation of the composition is selected from the following group: tablets, capsules, injections, granules, sprays, and lyophilized agents.

In another preferred embodiment, the dosage form of the composition is an injection.

In another preferred embodiment, the injection is intravenous injection, intramuscular injection or subcutaneous injection.

In another preferred embodiment, the polypeptide is administered to mammals at a dose of 0.01-100 mg/kg body weight (each time or every day).

In a seventh aspect of the present invention, there is provided uses of a polypeptide or a pharmaceutically acceptable salt thereof as described in the first aspect of the present invention, a fusion protein as described in the second aspect of the present invention, and a polynucleotide as described in the third aspect of the present invention, a vector as described in the fourth aspect of the present invention, a host cell as described in the fifth aspect of the present invention, and/or the composition as described in the sixth aspect of the present invention, which are used to prepare the composition, the composition is used for one or more uses selected from the following groups: (a) inhibiting TRPM8; (b) preventing and/or treating TRPM8-related diseases; (c) preventing and/or treating cold allodynia; (d) preventing and/or treating peripheral neuropathy; (e) preventing and/or treating pain; and/or (f) preventing and/or treating chronic constriction injury of neuropathic pain.

In another preferred embodiment, the composition is a pharmaceutical composition.

In another preferred embodiment, the TRPM8-related diseases are selected from the following group: peripheral neuropathy, cold allodynia, pruritus, chronic constriction injury of neuropathic pain, or combinations thereof.

In another preferred embodiment, the pain is selected from the following group: chronic pain, cold allodynia pain, neuropathic pain of diabetic neuropathy, postoperative pain, osteoarthritis pain, rheumatoid arthritis pain, Cancer pain, neuralgia, nerve damage pain, migraine, cluster headache, tension headache, fibromyalgia, neuropathic pain, static allodynia, cold allodynia, or combinations thereof.

In another preferred embodiment, the cold allodynia includes a cold allodynia caused by chronic constriction injury of neuropathic pain.

In another preferred embodiment, the static allodynia includes a static allodynia caused by chronic constriction injury of neuropathic pain.

In another preferred embodiment, the TRPM8-related diseases include TRPM8 upregulation diseases.

In another preferred embodiment, the TRPM8 upregulation includes high TRPM8 expression level or activity.

In another preferred example, the TRPM8 upregulation means that the TRPM8 expression level or activity of a certain cell (such as stimulation receptor neurons or dorsal root ganglia) is greater than that of the same cell.

In another preferred example, the TRPM8 upregulation means that the ratio of the TRPM8 expression level or activity C1 of a certain cell (such as stimulation receptor neurons or dorsal root ganglia) to the TRPM8 expression level or activity C0 of the same cell (C1/C0) is >1.0, preferably ≥1.2, preferably ≥1.5, more preferably ≥2, even more preferably ≥3, most preferably ≥5.

In another preferred embodiment, the cell include nerve cell.

In another preferred embodiment, the stimulation-sensitive neurons include nociceptive stimulation neurons.

In another preferred embodiment, the dorsal root ganglion includes nociceptive dorsal root ganglion.

In another preferred embodiment, the same cell refers to a cell of the same type and with normal expression or activity of TRPM8.

In another preferred embodiment, the expression includes mRNA and/or protein expression.

In another preferred embodiment, the cold allodynia includes cold allodynia induced by platinum anticancer drugs.

In another preferred embodiment, the peripheral neuropathy includes peripheral neuropathy caused by platinum anticancer drugs.

In another preferred embodiment, the cold allodynia includes cold allodynia induced by platinum anticancer drugs.

In another preferred embodiment, the platinum anticancer drug is selected from the following group: carboplatin, nedaplatin, loplatin, oxaliplatin, or a combination thereof.

An eighth aspect of the present invention provides a method of (a) inhibiting TRPM8; (b) preventing and/or treating TRPM8-related diseases; (c) preventing and/or treating cold allodynia; (d) preventing and/or treating peripheral neuropathy; (e) preventing and/or treating pain; and/or (f) preventing and/or treating chronic constriction injury of neuropathic pain, the method comprising the steps of: administering a polypeptide or a pharmaceutically acceptable salt thereof as described in the first aspect of the present invention, a fusion protein as described in the second aspect of the present invention, a polynucleotide as described in the third aspect of the present invention, a vector as described in the fourth aspect of the present invention, a host cell as described in the fifth aspect of the present invention, and/or a composition as described in the sixth aspect of the present invention to a subject in need.

In another preferred embodiment, the subject is a human or non-human mammal.

In another preferred embodiment, the non-human mammal include a rodent (such as mouse, rat, rabbit) and a primate (such as monkey).

In another preferred embodiment, the method is non-diagnostic and non-therapeutic.

In another preferred embodiment, the administration is oral administration, injection administration or external administration.

In another preferred embodiment, the injection is intravenous injection, intramuscular injection or subcutaneous injection.

It should be understood that within the scope of the present invention, the above-mentioned technical features of the present invention and the technical features specifically described below (such as embodiments) can be combined with each other to form new or preferred technical solutions. Due to space limitations, they will not be described one by one here.

### DESCRIPTION OF DRAWINGS

FIG. 1 shows the electrophysiological verification of HPLC purification, mass spectrometry verification and their functions of the designed polypeptide after synthesis. (a) shows HPLC purification of DeC-1.1 polypeptide. (b) shows mass spectrometry verification of the molecular weight of the DeC-1.1 peptide. (c) shows HPLC purification of DeC-1.2 polypeptide. (d) shows mass spectrometry verification of the molecular weight of the DeC-1.2 polypeptide. (e) shows representative curves of whole-cell electrophysiological recordings of DeC-1.2 inhibiting menthol-activated mouse TRPM8 channels. (f) shows inhibitory concentration-dependent curves of DeC-1.1 and DeC-1.2 measured in whole-cell electrophysiological recording mode.

FIG. 2 shows the specific inhibition of the activation mode of TRPM8 by DeC-1.2. (a) shows a representative inside-out single-channel electrophysiological recording of TRPM8 at +80 mV. TRPM8 channels are activated or inhibited by perfusion with saturated menthol (1 mM) and a mixed solution of saturated menthol (1 mM) and DeC-1.2 (100 μM), respectively. These four representative single-channel recordings are from the same piece of cell membrane. (b) shows a full-point histogram corresponding to the single-channel electrophysiological recording of TRPM8 shown in Fig. (a). The histogram is fitted with a double Gaussian function (represented by the solid red line). After fitting, the difference between two adjacent peaks is used to calculate the single-channel conductance. (c) shows that the open probability of TRPM8 channels activated by a saturating concentration of Menthol (1 mM) decreases significantly in the presence of DeC-1.2 (100 μM). Data are shown as the mean±SEM of electrophysiological recording data from five independent cells per group. **** indicates P<0.0001. (d) show that the single-channel conductance of TRPM8 channels activated by a saturating concentration of Menthol (1 mM) decreases significantly in the presence of DeC-1.2 (100 μM). Data are shown as the mean±SEM of electrophysiological recording data from three independent cells per group. * indicates P<0.001. (e) shows a representative inside-out single-channel electrophysiological recording of TRPM8 being cold-activated in the presence and absence of DeC-1.2 at +80 mV. (f) shows a full-point histogram corresponding to the TRPM8 single-channel electrophysiological recording shown in panel (e). The histogram is fitted using a double Gaussian function. (g) shows a that at 16° C., the open probability of TRPM8 channels being cold-activated does not change significantly in the presence of DeC-1.2 (100 μM). Data are shown as the mean±SEM of electrophysiological recording data from five independent cells per group. N.S. indicates no significant difference. (h) shows a that at 16° C., the single-channel conductance of cold-activated TRPM8 channels does not change significantly in the presence of DeC-1.2 (100 μM). Data are shown as the mean±SEM of electrophysiological recording data from three independent cells per group. N.S. indicates no significant difference.

FIG. 3 shows the subunit selectivity of DeC-1.2. (a, b, c, d, e) show that the representative electrophysiological recordings of DeC-1.2 inhibition of ligand activation of TRPV1, TRPV2, TRPV3, TRPM2 and TRPM4. Wherein the TRPM4-K1045A mutation is used to rule out the dependence of TRPM4 on PIP2. (f) shows the normalized comparison of the inhibitory effects of 500 nM DeC-1.2 on TRP channels and Nav channels. Each channel corresponds to n=3, **** means P<0.0001.

FIG. 4 shows the amino acid residues that play a key role in the inhibition of TRPM8 by DeC-1.2. (a, b, c, d, e, f, g) show the representative electrophysiological recordings of the inhibition of ligand activation of TRPM8 by 5 nM of wild-type DeC-1.2 and each mutant. (h, i) show normalized plots of inhibitory effects of 5 nM wild-type DeC-1.2 and mutants on ligand activation of TRPM8, *, ** and *** indicate P<0.05, P<0.01, P<0.001 respectively. (j) shows the amino acid residues indicated in the structure of DeC-1.2 that play a key role in the inhibitory effect of TRPM8 (mutations that completely eliminate inhibition and mutations that partially eliminate inhibition at 5 nM concentration are indicated in orange and orange, respectively).

FIG. 5 shows the validation of the inhibiting effect of DeC-1.2 on TRPM8 from an in vivo perspective. (a) shows DeC-1.2 dose-dependently significantly reduces the Icilin-induced wet dog-like whole-body tremor response in mice. Data are shown as means±SEM of six animals per group. *, ** and *** indicate p<0.05, p<0.01 and p<0.001 respectively. (b) shows a schematic diagram of the process of establishing oxaliplatin-induced cold hypersensitivity in mice and the flow chart of animal experiments on the inhibition of cold hypersensitivity by DeC-1.2. (c) shows representative graphs of calcium imaging responses when DRG neurons extracted from mice in the saline-treated group are perfused with extracellular fluid (Vehicle), when DRG neurons extracted from mice in the oxaliplatin-treated group are perfused with extracellular fluid (Oxaliplatin) or extracellular solution (Oxaliplatin+DeC-1.2) containing 10 μM DeC-1.2, respectively. After perfusion with 100 μM menthol, the calcium flux in DRG neurons increases as menthol activates TRPM8 channels. Calcium influx cells generated by activation of TRPV1 in DRG neurons by 1 μM capsaicin serves as a positive control. Neurons responding to agonists are indicated with white arrows. (d) shows representative curves of fluorescence ratios of DRG neurons respectively extracted from mice in the saline-treated and oxaliplatin-treated groups in response to the corresponding reagents. (e) shows the reaction proportion of DRGA neurons respectively extracted from mice in the saline-treated group and the oxaliplatin-treated group in response to menthol. Vehicle, oxaliplatin and oxaliplatin+DeC-1.2 correspond to the total number of neuronal cells in each group being 229, 238 and 334 respectively. *, ** represent P<0.05 and P<0.01 respectively. Each discrete circle represents the number of neurons in a field of view.

FIG. 6 shows the effect of DeC-1.2 on inhibiting oxaliplatin-induced cold allodynia in vivo. (a) shows the inhibiting effect of DeC-1.2 on oxaliplatin-induced cold allodynia. The total time of withdrawing, flinching or licking the paws (here are acetone-treated paws) within 5 minutes after acetone treatment is recorded as the acetone score to evaluate the inhibiting effect of DeC-1.2. Data are shown as means±SEM of six animals per group. With two-way ANOVA analysis method, * indicates p<0.05. (b) shows inhibiting effect of DeC-1.2 on oxaliplatin-induced mechanical touch allodynia. Data are shown as means±SEM from 5-8 mice per group. (c) shows the effect of DeC-1.2 on body temperature. The rectal temperature of each animal is measured before DeC-1.2 injection (30 μg/g, i.v.) (baseline, BL), and at 5 min, 15 min, 0.5 h, 1 h, 2 h, 6 h and 24 h after injection. Data are shown as means±SEM from six animals. After one-way ANOVA analysis, N.S. indicates no significant difference. (d) shows schematic diagram of the working principle of the activation mode-specific inhibitor DeC-1.2 on the TRPM8 channel.

### DETAILED DESCRIPTION OF EMBODIMENTS

After extensive and in-depth research, the inventors have prepared for the first time a polypeptide having uses of (a) inhibiting TRPM8; (b) preventing and/or treating TRPM8-related diseases; (c) preventing and/or treating cold allodynia; (d) preventing and/or treating peripheral neuropathy; (e) preventing and/or treating pain; and/or (f) preventing and/or treating chronic constriction injury of neuropathic pain, such as DeC-1.2 polypeptide, and the polypeptide of the invention is safe and has little toxic and side effects on biological tissues. On this basis, the inventor completed the present invention.

### Terms

Unless otherwise defined, all technical and scientific terms used herein have the same meaning as commonly understood by one of ordinary skill in the art to which this invention belongs.

As used herein, the terms “comprise,” “include” and “contain” are used interchangeably and include not only open definitions, but also semi-closed, and closed definitions. In other words, the terms include “consisting of” and “substantially consisting of.”

In the present invention, each amino acid and its abbreviation are shown in Table 1 below:

TABLE 1
Amino acid and its abbreviation

| | Three | Single | | Three | Single |
|---|---|---|---|---|---|
| | letter | letter | | letter | letter |
| Name | symbol | symbol | Name | symbol | symbol |
| alanine | Ala | A | leucine | Leu | L |
| arginine | Arg | R | lysine | Lys | K |
| asparagine | Asn | N | methionine | Met | M |
| asparticacid | Asp | D | phenylalanine | Phe | F |
| cysteine | Cys | C | proline | Pro | P |
| glutanine | Gln | Q | serine | Ser | S |
| gulatamic acid | Glu | E | threonine | Thr | T |
| glicine | Gly | G | tryptophan | Trp | W |
| histidine | His | H | tyrosine | Tyr | Y |
| isoleucine | Ile | I | valine | Val | V |

In the present invention, the term “preventing” means a method of preventing the onset of a disease and/or its accompanying symptoms or protecting a subject from acquiring a disease.

“Treating” as used in the present invention includes delaying and stopping the progression of the disease, or eliminating the disease, and does not require 100% inhibition, elimination, and reversal. In some embodiments, a polypeptide of the invention reduces, inhibits and/or reverses TRPM8-related disease, e.g., by at least about 10%, at least about 30%, at least about 50%, or at least about 80%, or 100% compared with levels observed in the absence of the polypeptide.

### TRPM8

TRPM8 (Transient receptor potential melastatin 8), also known as cold and menthol receptor, is a member of the transient receptor potential ion channel protein TRP family. The channel is composed of 4 identical subunits, each subunit has 6 transmembrane regions, and both N-terminus and C-terminus are located on the inside of the cell. The pore region located in the center of the TRPM8 channel is composed of four subunits and can non-selectively permeabilize cations. The TRPM8 channel is involved in the regulation of cold sensation, pain sensation, inflammatory response, vasoconstriction and expansion, cell growth and proliferation and like of the body.

### Polypeptide

In the present invention, the term “the polypeptide of the present invention” refers to a polypeptide having the structure shown in Formula I or a pharmaceutically acceptable salt thereof. It is understood that this term also includes mixtures of the above-mentioned components. In addition, the polypeptide of the present invention also include variant forms of the polypeptide having the structure shown in Formula I. These variant forms include (but are not limited to): adding one or several (usually within 5, preferably within 3, and more preferably within 2) amino acids at the N-terminus. For example, in the art, substitutions with amino acids having similar or similar properties generally do not alter the function of the protein. Adding one or more amino acids to the N-terminus usually does not change the structure and function of the protein. Furthermore, the term also includes monomeric and polymeric forms of the polypeptides of the invention or pharmaceutically acceptable salts thereof.

In the present invention, the amino acid sequences of the polypeptide are numbered from the N-terminus to the C-terminus.

The invention also includes active fragments, derivatives and analogs of the polypeptides of the invention. The polypeptide fragments, derivatives or analogs of the present invention may be (i) a polypeptide in which one or more conservative or non-conservative amino acid residues (preferably conservative amino acid residues) are substituted, or (ii) a polypeptide in which one or more conservative amino acid residues have a substituted group, or (iii) a polypeptide formed by fusing a polypeptide of the present invention with another compound (such as a compound that extends the half-life of the polypeptide, for example polyethylene glycol), or (iv) a polypeptide formed by additional amino acid sequence being fused with this polypeptide sequence (a subsequent protein formed by fusion with a tag sequence such as a leader sequence, a secretion sequence or 6His). Such fragments, derivatives and analogs are within the scope of those skilled in the art according to the teachings herein.

A preferred class of active derivatives refers to polypeptides that are formed by at most 5, preferably at most 3, more preferably at most 2, and most preferably 1 amino acid being substituted with the amino acid having similar or similar properties, compared to the amino acid sequence of the polypeptide shown in Formula I. These conservative variant polypeptides are preferably produced by amino acid substitutions according to Table 2.

TABLE 2

| original | representative | preferred |
|---|---|---|
| residues | substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; | Leu |
| | Phe | |
| Leu (L) | Ile; Val; Met; Ala; | Ile |
| | Phe | |
| Lys (K) | Arg; Gln; Asn | Arg |
| Met (M) | Leu; Phe; Ile | Leu |
| Phe (F) | Leu; Val; Ile; Ala; | Leu |
| | Tyr | |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala | |

The invention also provides analogs of the polypeptides of the invention. The difference between these analogs and the natural polypeptide of the present invention may be differences in amino acid sequence, differences in modified forms that do not affect the sequence, or both. Analogues also include analogs with residues that differ from natural L-amino acids (e.g., D-amino acids), as well as analogs with non-naturally occurring or synthetic amino acids (e.g., β, γ-acids). For example, Cys can form disulfide bonds with non-natural Hcy. It should be understood that the polypeptides of the present invention are not limited to the representative polypeptides exemplified above.

Modification forms (which generally do not change the primary structure) include chemically derivatized forms of the polypeptide in vivo or in vitro, such as acetylation or carboxylation. Modification also include glycosylation, such as the polypeptide resulting from glycosylation modification of the polypeptide during its synthesis and processing or during further processing steps. This modification can be accomplished by exposing the polypeptide to an enzyme that performs glycosylation, such as a glycosylase or deglycosylase of mammalian. The modification forms also include sequences having phosphorylated amino acid residues (e.g., phosphotyrosine, phosphoserine, phosphothreonine). Also included are polypeptides that have been modified to increase their resistance to proteolysis or to optimize solubility properties.

In preferred embodiments, the polypeptide of the present invention has at least one internal disulfide bond (introduced intrachain disulfide bond). Surprisingly, the presence of this internal disulfide bond not only does not affect its activity, but also contributes to extending the half-life and improving inhibitory activity. Generally, the disulfide bond can be formed by conventional methods in the art, such as combining the cysteine or homocysteine thiol under oxidative conditions.

A preferred polypeptide of the present invention includes polypeptide DeC-1.2. The amino acid sequence of polypeptide DeC-1.2 is the amino acid sequence described in SEQ ID No.: 1:

SEQ ID NO.: 1
CRRDRARHYRQRC.

In the present invention, the terms “DeC-1.2”, “polypeptide DeC-1.2” or “DeC-1.2 polypeptide” are used interchangeably.

The polypeptide of the present invention also include polypeptide modified for the polypeptide shown in SEQ ID NO.: 1.

The polypeptide of the invention may also be used in the form of salts derived from pharmaceutically or physiologically acceptable acids or bases. These salts include, but are not limited to, salts formed with the following acids: hydrochloric acid, hydrobromic acid, sulfuric acid, citric acid, tartaric acid, phosphoric acid, lactic acid, pyruvic acid, acetic acid, succinic acid, oxalic acid, fumaric acid, maleic acid acid, oxaloacetic acid, methanesulfonic acid, ethanesulfonic acid, benzenesulfonic acid, or isethionic acid. Other salts include salts formed with alkali metals or alkaline earth metals such as sodium, potassium, calcium or magnesium, and in the form of esters, carbamates or other conventional “prodrugs”.

### Coding Sequence

The invention also relates to polynucleotides encoding the polypeptides of the invention. The polynucleotides of the invention may be in DNA form or RNA form. DNA can be a coding strand or a non-coding strand. The coding region sequence encoding the mature polypeptide may be identical to the coding region sequence or may be a degenerate variant. The full-length nucleotide sequence of the polypeptide of the present invention or its fragment can usually be obtained by PCR amplification, recombination or artificial synthesis. At present, the DNA sequence encoding the polypeptide of the present invention (or its fragment, or its derivative) can be obtained entirely through chemical synthesis. The DNA sequence can then be introduced into a variety of existing DNA molecules (or vectors) and cells known in the art.

The present invention also relates to vectors comprising the polynucleotides of the invention, as well as host cells genetically engineered using the vectors of the invention or the polypeptide coding sequences of the invention.

On the other hand, the invention also includes polyclonal antibodies and monoclonal antibodies or antibody fragments, especially monoclonal antibodies, specific for the polypeptides of the invention.

In the context of two nucleic acids or polypeptides, when performing maximum identity sequence comparisons and alignments, the term “substantially identical” refers to two or more sequences or subsequences in which at least about 80%, such as at least about 85% %, about 90%, about 95%, about 98%, or about 99% of the nucleotide or amino acid residues are identical to a particular reference sequence, as determined using the following sequence comparison methods and/or by visual inspection.

### Preparation Method

The polypeptides of the invention may be recombinant polypeptides or synthetic polypeptides. The polypeptides of the present invention can be chemically synthesized or recombinant. Correspondingly, the polypeptide of the present invention can be artificially synthesized by conventional methods or produced by recombinant methods.

A preferred method is to use liquid phase synthesis technology or solid phase synthesis technology, such as Boc solid phase method, Fmoc solid phase method or a combination of the two methods. Solid-phase synthesis can quickly obtain samples, and appropriate resin vectors and synthesis systems can be selected according to the sequence characteristics of the target peptide. For example, the preferred solid phase vector in the Fmoc system is Wang resin connected to the C-terminal amino acid in the peptide. The structure of Wang resin is polystyrene, and the arm between the amino acid and the amino acid is 4-alkoxybenzyl alcohol; treating with 25% hexahydropyridine/dimethylformamide at room temperature for 20 minutes to remove the Fmoc protecting group, and extending from the C-terminus to the N-terminus one by one according to the given amino acid sequence. After the synthesis is completed, using trifluoroacetic acid containing 4% p-cresol to cut the synthesized proinsulin-related peptide from the resin and removing the protecting group, filtering the resin out and then being separated by diethyl ether precipitation to obtain the crude peptide. After lyophilizing the resulting product solution, filtering with gel and purifying the desired peptide by reversed-phase high-pressure liquid chromatography.

When using the Boc system for solid-phase synthesis, the preferred resin is PAM resin connected to the C-terminal amino acid in the peptide. The PAM resin structure is polystyrene, and the arm between the amino acid and the amino acid is 4-hydroxymethylphenylacetamide; in the Boc synthesis system, in the cycle of deprotection, neutralization and coupling, removing the protecting group Boc with TFA/dichloromethane (DCM) and neutralizing with diisopropylethylamine (DIEA/dichloromethane). After condensation of the peptide chain is completed, treating with hydrogen fluoride (HF) containing p-cresol (5-10%) at 0° C. for 1 hour to cut the peptide chain from the resin and removing the protecting group. Extracting the peptide with 50-80% acetic acid (containing a small amount of mercaptoethanol), and the solution is lyophilized and further separated and purified with molecular sieve Sephadex G10 or Tsk-40f, and then subjected to high-pressure liquid phase purification to obtain the desired peptide. Various amino acid residues can be coupled using various coupling agents and coupling method known in the peptide chemistry field, for example, dicyclohexylcarbodiimide (DCC), hydroxybenzotriazole (HOBt) or 1,1,3,3-tetraurea hexafluorophosphate (HBTU) can be used for directly coupling. For the synthesized short peptide, its purity and structure can be confirmed by reversed-phase high-performance liquid chromatography and mass spectrometry analysis.

In a preferred embodiment, the polypeptide of the present invention is prepared according to its sequence by a solid-phase synthesis method, and is purified by high-performance liquid chromatography to obtain a high-purity target peptide lyophilized powder, which is stored at −20° C.

Another method is to use recombinant technology to produce the polypeptides of the invention. By conventional recombinant DNA technology, the polynucleotides of the invention can be used to express or produce the recombinant polypeptides of the invention. Generally, there are the following steps:
(1). transforming or transducing appropriate host cells with the polynucleotide (or variant) encoding the polypeptide of the present invention, or with a recombinant expression vector containing the polynucleotide;
(2). culturing the host cells in appropriate culture media;
(3). separating and purifying proteins from culture media or cells.

The recombinant polypeptides can be expressed within cells or on cell membranes or secreted outside cells. If desired, the recombinant protein can be separated and purified by various separation methods utilizing its physical, chemical and other properties. These methods are well known to those skilled in the art. Examples of these methods include, but are not limited to: conventional renaturation treatment, treatment with protein precipitating agents (salting out method), centrifugation, osmotic sterilization, ultratreatment, ultracentrifugation, molecular sieve chromatography (gel filtration), adsorption layer analysis, ion exchange chromatography, high performance liquid chromatography (HPLC) and various other liquid chromatography techniques and combinations of these methods.

Since the polypeptide of the present invention is short, it is possible to connect multiple polypeptides in series together, obtain an expression product in the form of a polymer after recombinant expression, and then form the required small peptide through enzyme digestion or other methods.

### Composition and Administration Method

The present invention also provides a composition, which is preferably a pharmaceutical composition.

The composition of the present invention contains (a) the polypeptide of the present invention or a pharmaceutically acceptable salt thereof; and (b) a pharmaceutically acceptable vector or excipient. The amount of the polypeptide of the present invention or the pharmaceutically acceptable salt thereof is usually 10 micrograms-100 mg/dose, preferably 100-1000 micrograms/dose.

For the prevention and treatment purposes of the present invention, in the composition of the present invention, the polypeptide of the present invention or the pharmaceutically acceptable salt thereof is a safe and effective amount, and the effective dose is about 0.01 mg/kg to 50 mg administered to an individual/kg, preferably 0.05 mg/kg to 10 mg/kg body weight of the polypeptide of the present invention or a pharmaceutically acceptable salt thereof. In addition, the polypeptide of the present invention or a pharmaceutically acceptable salt thereof can be used alone or together with other therapeutic agents (such as formulated in the same pharmaceutical composition).

Pharmaceutical compositions may also contain pharmaceutically acceptable vectors. The term “pharmaceutically acceptable vector” refers to a vector used for the administration of a therapeutic agent. This term refers to pharmaceutical vectors that do not themselves induce the production of antibodies that are harmful to the individual receiving the composition and do not exhibit undue toxicity after administration. These vectors are well known to those of ordinary skill in the art. A thorough discussion of pharmaceutically acceptable excipients can be found in Remington's Pharmaceutical Sciences (Mack Pub. Co., N.J. 1991). Such vectors include, but are not limited to: saline, buffer, glucose, water, glycerol, ethanol, adjuvants, and combinations thereof.

Pharmaceutically acceptable vectors in therapeutic compositions may contain liquids such as water, saline, glycerin, and ethanol. In addition, these vectors may also contain auxiliary substances, such as wetting agents or emulsifiers, pH buffer substances, etc.

Generally, the therapeutic compositions may be prepared as injections, such as liquid solutions or suspensions; they may also be prepared in solid forms suitable for solution or suspension in liquid vectors prior to injection.

Once the compositions of the present invention are formulated, they may be administered by conventional routes, including but not limited to: intramuscular, intravenous, subcutaneous, intradermal, or topical administration. The subject to be prevented or treated can be an animal; especially a human.

In a preferred embodiment of the present invention, the dosage form of the pharmaceutical composition is an oral preparation, an injection preparation or an external preparation.

In a preferred embodiment of the present invention, the dosage form of the pharmaceutical composition is a solid preparation, a liquid preparation or a semi-solid preparation.

In a preferred embodiment of the present invention, the preparation of the pharmaceutical composition is selected from the following group: tablets, capsules, injections, granules, sprays, and lyophilized agents.

Typically, the injection is intravenous injection, intramuscular injection or subcutaneous injection.

When the pharmaceutical composition of the present invention is used for actual treatment, pharmaceutical compositions in various dosage forms can be used according to the usage conditions. Preferably, it is an intravenous pharmaceutical preparation or an intratumoral pharmaceutical injection.

These pharmaceutical compositions can be formulated by mixing, diluting or dissolving according to conventional methods, and occasionally adding suitable pharmaceutical additives such as excipients, disintegrants, binders, lubricants, diluents, buffers, isotonicities, preservatives, wetting agents, emulsifiers, dispersants, stabilizers and co-solvents, and the formulation process can be carried out in a conventional manner according to the dosage form.

For example, the formulation of eye drops can be carried out as follows: dissolving the polypeptide of the present invention or a pharmaceutically acceptable salt thereof together with basic substances in sterile water (surfactant is dissolved in sterile water), and adjusting the osmotic pressure and pH to physiological state, and optionally adding suitable pharmaceutical additives such as preservatives, stabilizers, buffers, isotonic agents, antioxidants and viscosifiers, and then completely dissolving.

The pharmaceutical composition of the present invention can also be administered in the form of a sustained-release preparation. For example, the polypeptides of the present invention or pharmaceutically acceptable salts thereof can be incorporated into pellets or microcapsules using sustained-release polymers as vectors, and the pellets or microcapsules are then surgically implanted into the tissue to be treated. Examples of sustained-release polymers include ethylene-vinyl acetate copolymer, polyhydrometaacrylate, polyacrylamide, polyvinylpyrrolidone, methylcellulose, lactic acid polymer, Lactic acid-glycolic acid copolymers and the like, preferably biodegradable polymers such as lactic acid polymers and lactic acid-glycolic acid copolymers can be exemplified.

The drug preparation should match the mode of administration. The drug preparation of the invention may also be used with other synergistic therapeutic agents (including before, during or after use). When a pharmaceutical composition is used, a safe and effective amount of the drug is administered to a desired subject (e.g., a human or a non-human mammal), which is usually at least about 10 micrograms per kilogram of body weight, and in most cases no more than about 8 mg/kg body weight, preferably the dosage is about 10 micrograms/kg body weight to about 1 mg/kg body weight. Of course, the specific dosage should also take into account factors such as the route of administration and the patient's health condition, which are all within the skill of a skilled physician.

The main effects of the present invention include:
(1) The polypeptide of the present invention can effectively (a) inhibit TRPM8; (b) prevent and/or treat TRPM8-related diseases; (c) prevent and/or treat cold allodynia; (d) prevent and/or treat Peripheral neuropathy; (e) prevention and/or treatment of pain; and/or (f) prevent and/or treat chronic constriction injury of neuropathic pain.
(2) The polypeptide of the present invention and its derivative polypeptides have small molecular weight, low toxic and side effects on biological tissues, and high safety.
(3) The polypeptide of the present invention has good stability.
(4) The polypeptide of the present invention has high specificity.

The present invention will be further described below in conjunction with specific embodiments. It should be understood that these examples are only used to illustrate the invention and are not intended to limit the scope of the invention. Experimental methods without specifying specific conditions in the following examples usually follow conventional conditions or conditions recommended by the manufacturer. Unless otherwise stated, percentages and parts are by weight.

### Embodiment 1

### Experiment Method:

### Synthesis and Purification of Peptides

The polypeptides with or without a disulfide bond are synthesized by GL Biochem (Shanghai) Ltd. Crude peptides were further purified by reverse-phase (RP) HPLC. Once the purification efficiency of the polypeptide was up to 95% or more, it could be identified and confirmed by matrix-assisted laser desorption/ionization time-of-flight mass spectrometry (MALDI-TOF) or electrospray ionization mass spectrometry (ESI-MS). After purification mass spectrometry identification, a large number of corresponding cyclic peptides were collected for backup.

### Cell Culture and Transient Transfection

HEK293T cells were cultured in Dulbecco's modified Eagle culture medium under a 37° C., 5% CO2 environment, 10% fetal bovine serum, penicillin (100 U/mL), and streptomycin (100 mg/mL) was needed to add into the culture medium. Transient transfection was conducted by using Lipo 2000 (Invitrogen) transfection kit and following the instruction manual.

### Electrophysiology Recording

Electrophysiological experiment recordings were performed on the cells within 24˜48 h of transient transfections. Specifically, single channel electrophysiological recordings were performed within 8 h after transfection.

Measurement of the cell currents was respectively performed in the whole-cell recording mode, inside-out recording mode or outside-out recording mode with a HEKA EPC10 amplifier. In the whole cell electrophysiological recordings, glass microelectrodes made of borosilicate material were fire-polished to a original electrode resistance of about 3-6 MΩ. Serial resistance was compensated by 60%. The whole cell electrophysiological recordings were performed at ±80 mV. For single channel electrophysiological recordings, glass microelectrodes were fire-polished to a higher resistance of 6-8 MΩ2. Current signal was sampled at 10 kHz and filtered at 2.9 kHz. Membrane potential of the cell membrane would be clamped at +80 mV. All electrophysiological recordings were performed at room temperature (22° C.) (the maximum variation is 1° C.).

There were different bath and pipette solutions prepared for different ion channels current measurement. For ion channels such as TRPM8, TRPV1, TRPV2, TRPV3 and TRPA1, the formula of the bath and pipette solutions was: 130 mM NaCl, 0.2 mM EDTA and 3 mM HEPES, pH=7.2. For the current measurement of TRPM4 ion channel, the formula of the bath solution was: 130 mM NaCl and 3 mM HEPES, pH=7.2, and the formula of the pipette solution was: 500 μM CaCl₂), 130 mM NaCl and 3 mM HEPES, pH=7.2. For the current measurement of TRPM2 ion channel, the formula of the bath solution was: 147 mM NaCl, 2 mM KCl, 1 mM MgCl₂, 10 mM HEPES, 2 mM CaCl₂) 13 mM Glucose, pH=7.4, and the formula of the pipette solution was: 147 mM NaCl, 1 mM MgCl₂, 10 mM HEPES, pH=7.4. For the current measurement of Naᵥ1.5, Naᵥ1.7 ion channels, the formula of the bath solution was: 140 mM NaCl, 3 mM KCl, 1 mM MgCl₂, 1 mM CaCl₂), 10 mM HEPES, pH=7.2 and the formula of the pipette solution was: 140 mM CsF, 1 mM EGTA, 10 mM NaCl, 10 mM MgCl₂, 3 mM KCl, pH=7.2. To evoke sodium channel current, the membrane potential was increased from −80 mV to +10 mV.

A rapid solution changer with a gravity-driven perfusion system (RSC-200, BioLogic) was used to rapidly perfuse specific solutions into the cells to be tested. Each solution was delivered through a separate tube so that there is no mixing of solutions. The glass microelectrode with a membrane patch was placed directly below the outlet of the perfusion tube during the electrophysiology recordings.

### Animal Experiment

Adult male CD1 mice (6-8 weeks old) were used as experimental animals for behavioral experiments and primary cultures of DRG neurons. Mice were raised in the condition of 12 h light/12 h dark of light cycle with adequate standard chow and water. All animal protocols were approved by the Institutional Animal Care and Use Committee (IACUC) of Zhejiang University.

### Mice Models of Pain

All of mice were habituated to the behavioral testing environment for 1-to-2 hours in 2-to-3 days before the behavioral testing. Single intraperitoneal (i.p.) injection of oxaliplatin (6 mg/kg body weight) was given to generate chemotherapy-induced neuropathic pain. Neuropathic pain behaviors were tested about 10 days after oxaliplatin injection.

### Icilin-Induced Wet-Dog Shake Test

At least 6 adult male CD1 mice were prepared for experimental or control groups. The animals were pretreated with Vehicle or DeC-1.2 of gradient dose via intravenous injection (i.v.) at 30 min before icilin (intraperitoneal injection, i.p., 2.5 μg/g body weight) treatment. Wet dog shake behavior was recorded and counted for 30 min following icilin administration.

### Mice Body Temperature Measurement

A digital thermometer (FT3400) was used for body temperature measurement of mice. Adult CD1 male mice were placed into an environmental room maintained at a constant temperature of 22±1° C. Mice were injected with DeC-1.2 (30 mg/kg body weight) via the tail vein to assess the effect of DeC-1.2 on body temperature. Body temperature was measured before treatment (baseline, BL) and at 5 min, 15 min, 0.5, 1, 2, 6 and 24 hours following DeC-1.2 (30 mg/kg, i.v.) administration. The test probe of the thermometer was inserted into the anus of the mouse approximately 2 cm, and the body temperature was recorded after the temperature display value was stable.

### Oxaliplatin-Induced Cold Allodynia

Acetone-induced evaporating cold test was used to assess the oxaliplatin-induced cold allodynia. 50 μL acetone was gently splashed to the hind-paw plantar of oxaliplatin-treated mice via a syringe attached with plastic tube. The time spent in withdrawing, flinching or licking the stimulated paw was recorded and blindly counted during 5 min after acetone treatment. The cold allodynia behavior of mice was respectively measured on day 10 pre- or post-treatment with oxaliplatin, Vehicle or 3.5 μg/20 μL DeC-1.2 was intraplantarly injected to assess its analgesic effect on oxaliplatin-induced allodynia.

### Oxaliplatin-Induced Mechanical Allodynia

Mechanical allodynia was assessed by measuring paw withdrawal thresholds in von Frey test. The paw withdrawal thresholds of mice in the von Frey test were tested according to Dixon'sup-down method. The middle of the hind-paw plantar of mice was stimulated by von Frey hairs (0.02-2 g). Mice showed withdrawing, flinching and licking their paws within 3 s of stimulation, i.e. responded. According to Dixon's up-down method, mice were first simulated with a 0.16 g vonFrey hair, then simulated with adjacent lower stiffness vonFrey hair if mice responded, or simulated with adjacent higher stiffness vonFrey hair if mice didn't respond. The vonFrey hairs with different stiffness were selected in turn to simulate, with 5 s interval per two simulations, and total six simulations were completed, the paw withdrawal thresholds (PWT) of mice were assessed according to the responding amount table The von Frey test was respectively performed on day 10 pre- or post-treatment with Oxaliplatin, saline or 3.5 μg/20 μL DeC-1.2 was intraplantarly injected to assess its analgesic effect on oxaliplatin-induced allodynia.

### Primary Culture and Calcium Imaging Experiments of Mice DRG Neurons.

Mice was deeply anaesthetized, decapitated and blood was removed, DRG tissue was dissected off quickly and placed in ice-cold PBS solution. After washing the blood, digestive enzyme (Collagenase A 20 mg/100 ml, Dispase II 300 mg/100 ml dissolved in PBS) was added and DRG was digested at 37° C. for 1 hour. The samples were centrifuged at 500 g for 5 min and the digested tissue was collected. When the enzyme solution was removed, an appropriate amount of DMEM medium (containing 10% FBS, 1× Pen/Strep) was added, and the single cell suspension was prepared by mechanical trituration. The samples were centrifuged at 500 g for 10 min. Removing supernatant, and adding appropriate volume of Neurobasal medium (containing 2% B27,1 mML-Glutamine, 50 ng/ml NGF2.5S, 1× Pen/Strep), cells were resuspended and triturated into single cell suspension. The single suspension was inoculated on glass coverslips coated with poly-D-Lys. After cells adherence, adding appropriate medium to the culture dish, and DRG neurons were maintained in 37° C. incubator with 5% CO2 for 24 h before calcium imaging experiments.

The primary-cultured DRG neurons were washed with a extracellular solution (ECS, 140 mM NaCl, 5 mM KCl, 1 mM MgCl₂, 1.8 mM CaCl₂), 10 mM Glucose, 15 mM HEPES, pH=7.4) then incubated in calcium dye incubation solution (2 μM Fluo-2 AM, 0.05% Pluronic F-127 in ECS) for 30 min. The cell images were continuously and alternately excited by a high-speed continuous monochromatic light source under the excitation wavelength of 340 nm and 380 nm, and recorded in full vision under 20× object lens by Visiview software Intracellular fluorescence intensity changes were continuously recorded at 0.5 fps via a high-speed scanning camera Flash4.0LT. The ratio of fluorescence signal at 340 nm/380 nm represents the intensity of calcium signal. Menthol, DeC-1.2 (10 μM) and Capsaicin were applied via the fast exchange perfusion system (ALA-VM8; ALA Scientific Instruments) for 30 s, 90 s, and 30 s, respectively. The effect of DeC-1.2 on TRPM 8 channel activity was assessed by comparing the intensity of menthol-induced calcium signal and the proportion of positive reaction cells in DRG neurons from different group . . .

### Data Statistics

All experiments have been independently repeated for at least three times. All statistical data are given as mean±sem. Electrophysiology data were analyzed using paired or unpaired t-test. Behavioral experiment data were analyzed using t-test, one-way or two-way ANOVA. N.S. indicates no significance. *, **, *** and indicate p<0.05, p<0.01, p<0.001 and 0.0001, respectively.

### 1. Polypeptide Design and Chemical Synthesis

The entire amino acid sequence is RRDRARHYROR, and to further improve the stability of the designed peptide chain, we introduced a cysteine residue at its N-terminus and C-terminus respectively to form a disulfide bond, and the amino acid sequence of polypeptide DeC-1.2 is CRRDRARHYRQRC (SEQ ID NO: 1), and the polypeptide DeC-1.2 was cyclized.

The amino acid sequence of polypeptide DeC-1.1 is CRNSRAAHDSQKC (SEQ ID NO: 2), and the cysteine residues at the N-terminus and C-terminus of polypeptide DeC-1.1 form a disulfide bond, cyclizing polypeptide DeC-1.1. Meanwhile, a negative control polypeptide S-DeC-1.2 (SEQ ID NO:3) was designed.

The amino acids in both polypeptide DeC-1.2 and polypeptide DeC-1.1 were L-amino acids.

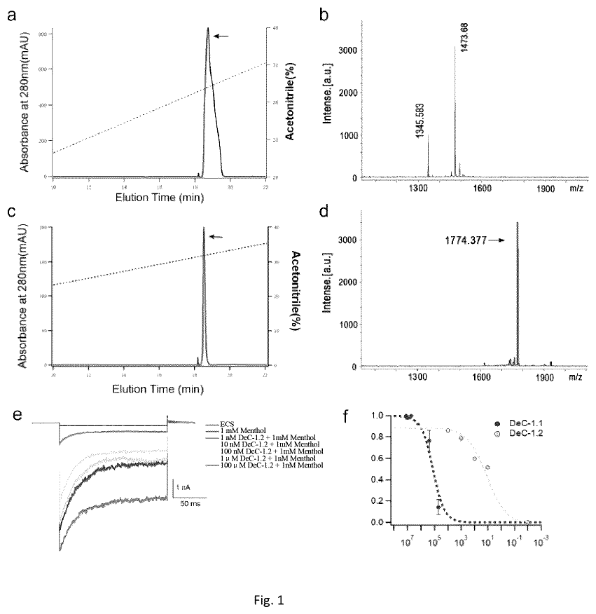

HPLC purification, mass spectrometry verification and electrophysiological validation of their function of the polypeptides after synthesis are shown in FIG. 1.

Polypeptide DeC-1.1 was chemically synthesized, purified by HPLC (FIG. 1a), identified and confirmed by mass spectrometry (FIG. 1b), and electrophysiologically tested, a half-inhibitory concentration (IC50) of DeC-1.1 was 132.8±73.5 μM.

Polypeptide DeC-1.2 was similarly chemically synthesized, purified by HPLC (FIG. 1c) and identified and confirmed by mass spectrometry (FIG. 1d). Upon electrophysiological testing, the polypeptide DeC-1.2 was able to inhibit Menthol activation of TRPM8 at the nanomolar level (FIG. 1e), showing a much lower half-inhibitory concentration (IC50), with polypeptide DeC-1.2 inhibiting TRPM8 at a half-inhibitory concentration (IC50) of 4.5±3.0 nM (FIG. 1b). In contrast, the negative control S-DeC-1.2 did not inhibit TRPM8.

The concentration-dependent curves of inhibitory properties of polypeptide DeC-1.1 and polypeptide DeC-1.2 measured in whole-cell electrophysiological recording mode are shown in FIG. 1f.

### 2. Characterization and Effects of Polypeptides

2.1 Modality- and Subunit-Specific Inhibition of TRPM8 Activation by polypeptideDeC-1.2

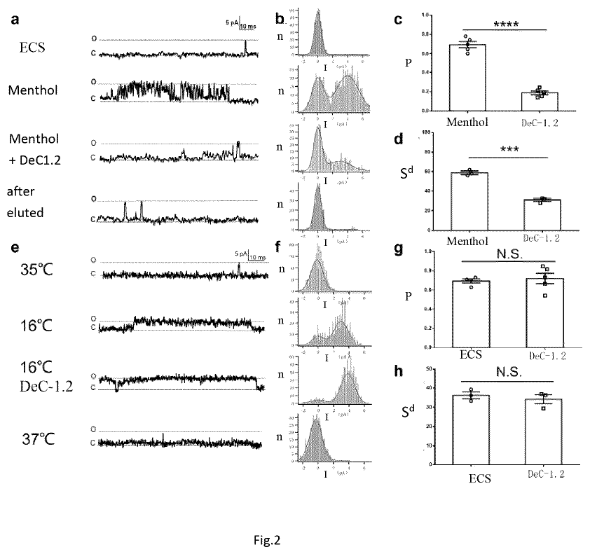

As polypeptideDeC-1.2 inhibited menthol activation of TRPM8 with high affinity, we next investigated whether the DeC-1.2 polypeptide showed modality-specific inhibition. The activation pattern-specific inhibition of TRPM 8 by DeC-1.2 is shown in FIG. 2. We performed single-channel electrophysiological recording, which allowed us to measure both the open probability and conductance of TRPM8 channel. The maximum open probability of TRPM8 activation by saturating concentration of menthol (1 mM) was about

70% (FIG. 2a-to-2c) [10]. When DeC-1.2 of 100 μM was applied with the saturated menthol (1 mM) to TRPM8 channel, the open probability was significantly reduced to 18.83±1.92% (n=5) (FIG. 2c). Moreover, we observed that the single-channel conductance of TRPM8 was also decreased by DeC-1.2 from 58.83±1.85 pS (n=3) to 30.97 #1.16 pS (n=3) (FIGS. 2b and 2d), suggesting that DeC-1.2 acted as a pore blocker to interfere the ion permeation in TRPM8.

We further examined whether DeC-1.2 was sufficient to inhibit the cold activation of TRPM8. When the membrane to be tested was cooled from 35° C. to 16° C., TRPM8 was activated. The application of DeC-1.2 (100 μM) at 16° C. did not decrease the open probability of TRPM8 (FIG. 2e-to-2g). Furthermore, though the single-channel conductance of TRPM8 was decreased to 36.16±1.74 pS (n=3) upon cooling, DeC-1.2 did not further reduce the single-channel conductance, suggesting that DeC-1.2 was an activation mode-specific inhibitor of TRPM 8. The single-channel conductance of the TRPM 8 channels activated by cold had no significant change in the presence of DeC-1.2 (100 μM) (FIG. 2h).

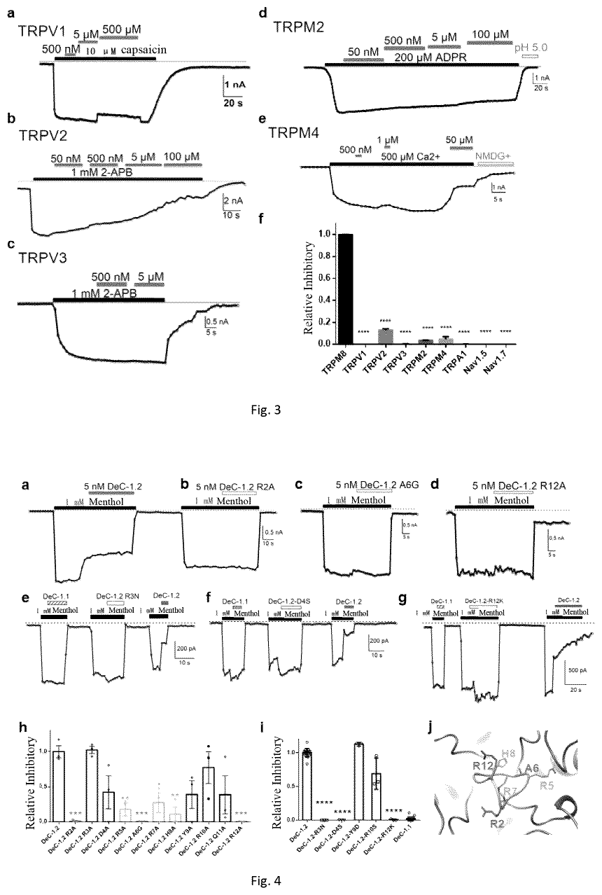

We further investigated the subunit selectivity of DeC-1.2. TRPM8 ion channel belongs to the TRP channel super family, the member channels of which all had a similar six transmembrane domain monomers. DeC-1.2 inhibited TRPM8 channel with an IC₅₀ value of 4.5 nM, however, a hundred-times higher concentration of DeC-1.2 (5 μM) failed to inhibit ligand activation of TRPV 1 or TRPV 3 (FIGS. 3a, 3b and 3c). Although DeC-1.2 inhibited TRPV2 channel to some extent, its half-inhibitory concentration (IC₅₀) was about 10 μM (FIG. 3b and Table 3), and inhibition on TRPV2 was far less efficient than on TRPM8. Among the TRP channels, TRPM2 is the closest homologue of TRPM8, but ligand activation of TRPM2 was not blocked by DeC-1.2 up to 100 μM (FIG. 3d). When we designed DeC-1.2, we used the structure of TRPM4 as the template for homology modeling of TRPM8 in the closed state, so DeC-1.2 may exhibit higher affinity for TRPM4. However, we found that TRPM4 showed much lower sensitivity to DeC-1.2, where 50 M of DeC-1.2 inhibited approximately half the current of TRPM4 (FIG. 3e). We also tested DeC-1.2 on TRPA1 and voltage-gated sodium (Naᵥ) channels and observed that none of the TRP channels and Naᵥ channels were inhibited by DeC-1.2 (Table 3). Therefore, DeC-1.2 is both a subunit-specific inhibitor and a modality-specific inhibitor to the ligand activation of TRPM8 channel.

TABLE 3
Inhibitory effects of DeC-1.2 peptide on different ion channels

| Ion channels | IC 50 | |
|---|---|---|
| TRPM8 | 4.5 ± 3.0 nM | |
| TRPV1 | >100 | μM |
| TRPV2 | >5 | μM |
| TRPV3 | >100 | μM |
| TRPM2 | >100 | μM |
| TRPM4 | >10 | μM |
| TRPA1 | >100 | μM |
| Nav1.5 | >100 | μM |
| Nav1.7 | >100 | μM |

Mutations (alanine scanning) of the DeC-1.2 polypeptide are performed as shown in Tables 4 and 5. These polypeptide with a single-point mutation were chemically synthesized and purified as wildtype DeC-1.2. The study of the amino acid residues with a critical role in the inhibition of TRPM 8 by DeC-1.2 was shown in FIG. 4. In whole-cell electrophysiological recordings mode, we observed that compared to the inhibitory activity of the wildtype DeC-1.2 at 5 nM, some mutants were still able to activate TRPM8, but there was a decrease in activity, while the inhibitory effect of some mutant cyclic peptides of 5 nM on TRPM8 was almost abolished (FIG. 4a, FIG. 4b, FIG. 4c, FIG. 4d, FIG. 4e, FIG. 4f, FIG. 4g), but at high concentrations, for example 100 μM, it also had the effect of inhibiting TRPM 8 channels. The key residues in the DeC-1.2 structure that were able to alter the inhibition of TRPM 8 by the cyclic peptide DeC-1.2 were further marked (FIG. 4h, FIG. 4i, FIG. 4j, mutations that completely abolished inhibition and partially inhibited were shown in red and orange, respectively). Most of the key residues were located at the interface between DeC-1.2 and the outer pore of TRPM8, and the mutations at these sites would significantly affect the inhibition cyclic peptide, suggesting that DeC-1.2 would bind to the out pore of TRPM 8 and play an inhibitory role.

TABLE 4
Mutant peptides based on DeC-1.2 peptide

| Peptide | Primary sequence |
|---|---|
| DeC-1.2 | CRRDRARHYRQRC (SEQ ID NO: 1) |
| S-DeC-1.2 | CDHRRRQARYRRC (SEQ ID NO: 3) |
| DeC-1.2 R2A | CARDRARHYRQRC |
| | (SEQ ID NO: 4) |
| DeC-1.2 R3A | CRADRARHYRQRC |
| | (SEQ ID NO: 5) |
| DeC-1.2 D4A | CRRARARHYRQRC |
| | (SEQ ID NO: 6) |
| DeC-1.2 R5A | CRRDAARHYRQRC |
| | (SEQ ID NO: 7) |
| DeC-1.2 A6G | CRRDRGRHYRQRC |
| | (SEQ ID NO: 8) |
| DeC-1.2 R7A | CRRDRAAHYRQRC |
| | (SEQ ID NO: 9) |
| DeC-1.2 H8A | CRRDRARAYRQRC |
| | (SEQ ID NO: 10) |
| DeC-1.2 Y9A | CRRDRARHARQRC |
| | (SEQ ID NO: 11) |
| DeC-1.2 R10A | CRRDRARHYAQRC |
| | (SEQ ID NO: 12) |
| DeC-1.2 Q11A | CRRDRARHYRARC |
| | (SEQ ID NO: 13) |
| DeC-1.2 R12A | CRRDRARHYRQAC |
| | (SEQ ID NO: 14) |

TABLE 5
Mutant peptides based on DeC-1.2 peptide

| Peptide | Primary sequence |
|---|---|
| DeC-1.1 | CRNSRAAHDSQKC ( SEQ ID NO: 2) |
| DeC-1.2 | CRRDRARHYRQRC (SEQ ID NO: 1) |
| DeC-1.2 R3N | CRNDRARHYRQRC |
| | (SEQ ID NO: 15) |
| DeC-1.2 D4S | CRRSRARHYRQRC |
| | (SEQ ID NO: 16) |
| DeC-1.2 Y9D | CRRDRARHDRQRC |
| | (SEQ ID NO: 17) |
| DeC-1.2 R10S | CRRDRARHYSQRC |
| | (SEQ ID NO: 18) |
| DeC-1.2 R12K | CRRDRARHYRQKC |
| | (SEQ ID NO: 19) |

### 2.2 Polypeptide Ameliorates Peripheral Neuropathy

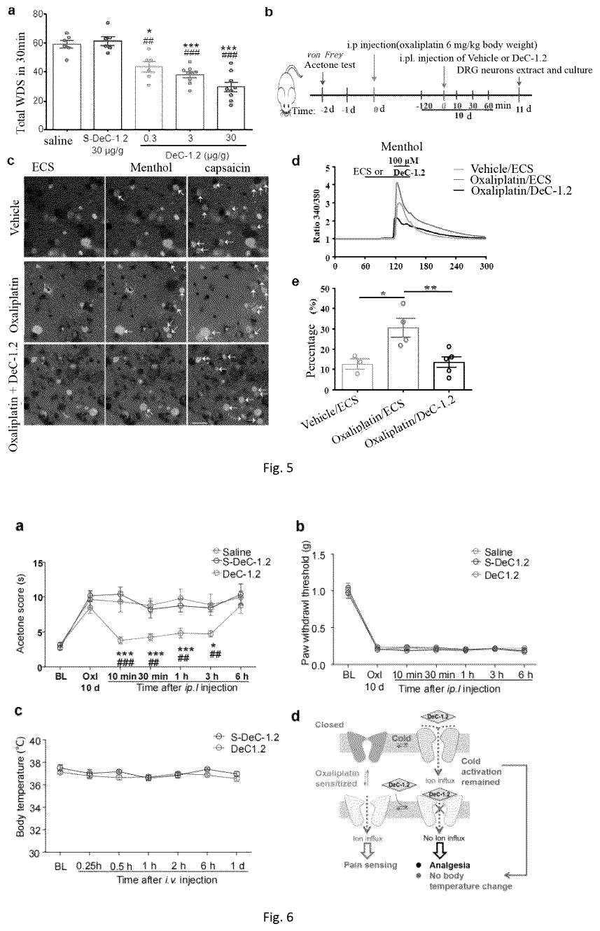

The wet-dog shake (WSD) behavior model induced by icilin (a potent agonist of TRPM8 channel) was used to evaluate the effect of polypeptide DeC-1.2 in mice, and the inhibitory effect of DeC-1.2 on TRPM 8 was shown in FIG. 5a. The results showed that intravenous injection of a low dose of DeC-1.2 (0.3 μg/g body weight) could significantly inhibit the WSD behaviors induced by icilin, and its inhibitory effect was significantly dose-dependent. The results showed that in vivo injection of DeC-1.2 could block TRPM 8 channel opening induced by Icilin.

Oxaliplatin will induce an increase in the activity of TRPM8 in nociceptive DRG neurons, thereby making DRG neurons more sensitive. To test whether DeC-1.2 could affect oxaliplatin-induced cold allodynia, we established the oxaliplatin-induced cold allodynia mice model (FIG. 5b). A single dose of oxaliplatin (6 mg/kg body weight) was intraperitoneally (i.p.) injected in mice on day 0; on day 10 when cold allodynia was fully developed, we examined the effect of polypeptide DeC-1.2 on the oxaliplatin-induced cold allodynia and the activity of TRPM8 in primary sensory neurons by behavioral testing and calcium imaging. At the cellular level, the dorsal root ganglion (DRG) neurons dissociated from the oxaliplatin-induced cold pain sensitivity model mice and normal mice (pointed out by arrows in white, FIG. 5c), and primary cultures of the neurons were performed. Calcium imaging experiments of primary cultured DRG neurons showed that in the control group (Vehicle injection group), the menthol could activate the calcium activity in neurons caused by TRPM8; compared to the control group, in the DRG neurons of oxaliplatin treated mice, both the amplitude of calcium influx induced by menthol activation of TRPM8 channel and the percentage of positive reaction cells were significantly increased; and DeC-1.2 could significantly inhibit the amplitude of calcium influx and the percentage of positive reaction cells in DRG neurons in Oxaliplatin treated mice (FIGS. 5d and 5e). These results showed that the DeC-1.2 polypeptide suppresses the activity of TRPM 8 channels in an oxaliplatin-induced cold allodynia model.

Furthermore, DeC-1.2 was intraplantarly (i.pl.) injected to test its effect on cold allodynia induced by acetone evaporation, the effect of DeC-1.2 on inhibiting oxaliplatin-induced cold allodynia in vivo was shown in FIG. 6. The results of the behavioral tests showed that ten days after oxaliplatin injection, the reaction time of the cold pain behavior caused by acetone (such as withdrawing, licking, and flinching the paw) increased significantly; the cold pain behavior of the control group (intraplantarly injected Vehicle) mice did not change significantly before and after intraplantar injection; and the intraplantar injection of 3.5 μg/20 μL DeC-1.2 significantly improved the cold pain behavior induced by acetone evaporation (FIG. 6a). In addition, the magnitude of analgesic effect against cold allodynia by DeC-1.2 application was similar to that of genetically knocking out of TRPM8 channel. In addition, in mechanical allodynia behavior tests, ten days after oxaliplatin injection, the paw withdrawal threshold of mice in the von Frey test significantly decreased, the same i.pl. injection of DeC-1.2 (3.5 μg/20 μl) did not change paw withdrawal threshold as compared to saline injection (FIG. 6b). More importantly, we observed that intravenous (i.v.) injection of DeC-1.2 at 30 μg/g body weight, which was high enough to largely reduce WDS behavior in icilin treated mice (FIG. 6a), did not significantly alter body temperature in mice (FIG. 6c). The working principle of the activation pattern-specific inhibitor DeC-1.2 on TRPM 8 channels was shown in FIG. 6d. Therefore, the cyclic peptide DeC-1.2 was able to improve cold allodynia behavior of mice through the activity of TRPM 8 channel without affecting body temperature of mice.

### 2.3 a Model of Chronic Constriction Injury (CCI) Induction of Neuropathic Pain: Cold Allodynia

Male Sprague-Dawley rats (purchased from Shanghai SLAC Laboratory Animal Co., Ltd. at the beginning of the experiment for 7 weeks, n=7-10/treatment) were used. CCI status was formed according to the method of Bennett G J and Xie Y K (Pain 1988, 33:87-107). Rats were anesthetized by intraperitoneal injection of sodium pentobarbital. The left common sciatic nerve was made to be exposed in the middle of the thigh in a state deprived of adherent tissue, and 4-0 silk thread (Ethicon Inc.) was utilized around it and four passes were loosely ligated at approximately 1-mm intervals. Sham surgery was performed in the same manner except for sciatic nerve ligation. After 1-2 weeks after CCI surgery, as documented in Tanimoto-Mori Setal. (BehavPharmacol., 19:85-90, 2008), The cold allodynia was evaluated by using a cooling plate (LHP-1700CP, TECA) with a temperature controller (Mode 13300-0, CAL Controls Inc.). The animals were acclimatized to a device consisting of a transparent acrylic box (10×12×12 cm) on a stainless steel plate (15×33 cm). The surface of the cooling plate was maintained at 10° C., and the temperature of the plate was continuously monitored with an accuracy of 0.1° C. To perform the experiment, rats were loaded on the cooling plate and the polypeptide DeC-1.2 was administered before and after a cutoff value of 120 seconds, and the paw withdrawal latency (PWL) was measured. Polypeptide DeC-1.2 or its mediator was administered orally, subcutaneously, or intraperitoneally. The inhibition rate (%) was [PWL (polypeptide)−PWL (mediator)]/[PWL (polypeptide)−PWL (mediator)]×100.

The results showed that the polypeptide DeC-1.2 exhibited strong activity in this model, with excellent results in the treatment of cold allodynia of chronic constriction injury of neuropathic pain.

### 2.4 Chronic Constriction Injury (CCI) Induction Model of Neuropathic Pain: Static Allodynia

Male Sprague Dawley rats (purchased from Shanghai SLAC Laboratory Animals, LLC, 7 weeks old at the beginning of the experiment, n=7-10 per treatment) were used. CCI status was formed according to the method of Bennett G J and Xie Y K (Pain 1988, 33:87-107). Rats were anesthetized by intraperitoneal injection of sodium pentobarbital. The left common sciatic nerve was made to be exposed in the middle of the thigh in a state deprived of adherent tissue, and 4-0 silk thread (Ethicon Inc.) was utilized around it and four passes were loosely ligated at intervals of approximately 1 mm. Sham surgery was performed in the same manner except for sciatic nerve ligation. CCI surgery was performed after 2-3 weeks as described in Field M J et al. (Pain 1999, 83:303-311) documented, static allodynia was evaluated by Von Frey Hairs (VFH). Before starting the experiment, the experimental animals were acclimatized to the grid bottom cage. The VFH was pressed against the hind-paw plantar, and the experiments were performed in a manner that gradually increased the force (0.16 g, 0.4 g, 0.6 g, 1 g, 1.4 g, 2 g, 4 g, 6 g, 8 g, 10 g, 15 g, and 26 g). Each VFH was pressed against the phasic paw for 6 seconds, or until a withdrawal response occurred. When one withdrawal response occurred, the experiment was repeated by pressing the VFH at a weaker pressure until no withdrawal response occurred. The minimum pressure required to induce a response was recorded as a withdrawal paw threshold (PWT). If the experimental animal responded below an innocuous 1.4 g VFH, it was determined to be in static allodynia. The polypeptide DeC-1.2 or its mediator was administered by injection, orally, subcutaneously, or intraperitoneally. The inhibition rate (%) was

[PWL(polypeptide)−PWL(mediator)]/[PWL (polypeptide)−PWL (mediator)]×100.

The results showed that the polypeptide DeC-1.2 exhibited strong activity in this model, with excellent results in the treatment of static allodynia of chronic constriction injury of neuropathic pain.

The foregoing is an embodiment of the present invention designed for one case, and it should be noted that for a person of ordinary skill in the art, a number of improvements can be made without departing from the principles of the present invention, and these improvements should also be regarded as the scope of protection of the present invention.

1. A polypeptide or a pharmaceutically acceptable salt thereof, wherein the polypeptide or the pharmaceutically acceptable salt thereof has a structure represented by Formula I:
X1-X2-X3-X4-X5-X6-X7-X8-X9-X10-X11-X12-X13 Formula I
in the formula,
X1 is none, any amino acid or any peptide fragment;
X2 is R, K, Q, A, D or N;
X3 is R, K, Q, A, H or N;
X4 is D, A, S, R or E;
X5 is R, K, Q, A or N;
X6 is A, V, L, G, R or I;
X7 is R, K, Q, A or N;
X8 is H, N, Q, K, A or R;
X9 is Y, W, F, D, T, A, R or S;
X10 is R, K, Q, A, S, Y or N;
X11 is Q, A, R or N;
X12 is R, K, Q, A, K or N;
X13 is none, any amino acid or any peptide fragment.

2. The polypeptide or the pharmaceutically acceptable salt thereof according to claim 1, wherein the polypeptide is selected from the following groups:
(1) a polypeptide that has an amino acid sequence shown in: SEQ ID NO: 1, SEQ ID NO: 2, SEQ ID NO: 4, SEQ ID NO: 5, SEQ ID NO: 6, SEQ ID NO: 7, SEQ ID NO: 8, SEQ ID NO: 9, SEQ ID NO:10, SEQ ID NO:11, SEQ ID NO:12, SEQ ID NO:13, SEQ ID NO:14, SEQ ID NO: 15, SEQ ID NO:16, SEQ ID NO: 17, SEQ ID NO: 18 or SEQ ID NO:19;
(2) a polypeptide that is formed by substitution, deletion or addition of 1-5 (preferably 1-3, more preferably 1-2) amino acid residues in the amino acid sequence shown in SEQ ID NO:1, SEQ ID NO:2, SEQ ID NO:5, SEQ ID NO:6, SEQ ID NO:7, SEQ ID NO:9, SEQ ID NO:10, SEQ ID NO: 11, SEQ ID NO: 12, SEQ ID NO: 13, SEQ ID NO: 15, SEQ ID NO: 16, SEQ ID NO: 17, SEQ ID NO: 18 or SEQ ID NO: 19 and has uses of: (a) inhibiting TRPM8; (b) preventing and/or treating a TRPM8-related disease; (c) preventing and/or treating cold allodynia; (d) preventing and/or treating peripheral neuropathy; (e) preventing and/or treating pain; and/or (f) preventing and/or treating chronic constriction injury of neuropathic pain;
(3) a polypeptide that has ≥50%, ≥60%, ≥70%, ≥80%, ≥90%, ≥95%, ≥99% or 100% homology (or homogeny) compared with a polypeptide shown in SEQ ID NO:1, SEQ ID NO:2, SEQ ID NO:5, SEQ ID NO:6, SEQ ID NO:7, SEQ ID NO:9, SEQ ID NO: 10, SEQ ID NO: 11, SEQ ID NO:12, SEQ ID NO: 13, SEQ ID NO: 15, SEQ ID NO:16, SEQ ID NO:17, SEQ ID NO: 18 or SEQ ID NO: 19.

3. A fusion protein, wherein the fusion protein comprises:
(a) the polypeptide or the pharmaceutically acceptable salt thereof according to claim 1;
(b) a peptide fragment fused to the polypeptide or the pharmaceutically acceptable salt thereof according to claim 1.

4. A polynucleotide, wherein the polynucleotide encodes the polypeptide or the pharmaceutically acceptable salt thereof according to claim 1.

5. A vector, wherein the vector comprises the polynucleotide according to claim 3.

6. A host cell, wherein the host cell comprises the vector according to claim 4.

7.-10. (canceled)

11. A host cell, wherein the host cell comprises the polynucleotide according to claim 3 is integrated into the chromosome of the host cell.

12. A composition, wherein the composition comprises:
(a) the polypeptide or the pharmaceutically acceptable salt thereof according to claim 1; and
(b) a pharmaceutically acceptable vector or excipient.

13. A use of the polypeptide or a pharmaceutically acceptable salt thereof according to claim 1, wherein it is used to prepare a composition, which is used for one or more uses selected from the following groups: (a) inhibiting TRPM8; (b) preventing and/or treating a TRPM8-related disease; (c) preventing and/or treating cold allodynia; (d) preventing and/or treating peripheral neuropathy; (e) preventing and/or treating pain; and/or (f) preventing and/or treating chronic constriction injury of neuropathic pain.

14. A use of the fusion protein according to claim 3, wherein it is used to prepare a composition, which is used for one or more uses selected from the following groups: (a) inhibiting TRPM8; (b) preventing and/or treating a TRPM8-related disease; (c) preventing and/or treating cold allodynia; (d) preventing and/or treating peripheral neuropathy; (e) preventing and/or treating pain; and/or (f) preventing and/or treating chronic constriction injury of neuropathic pain.

15. A use of the polynucleotide according to claim 4, wherein it is used to prepare a composition, which is used for one or more uses selected from the following groups: (a) inhibiting TRPM8; (b) preventing and/or treating a TRPM8-related disease; (c) preventing and/or treating cold allodynia; (d) preventing and/or treating peripheral neuropathy; (e) preventing and/or treating pain; and/or (f) preventing and/or treating chronic constriction injury of neuropathic pain.

16. A use of the vector according to claim 5, wherein it is used to prepare a composition, which is used for one or more uses selected from the following groups: (a) inhibiting TRPM8; (b) preventing and/or treating a TRPM8-related disease; (c) preventing and/or treating cold allodynia; (d) preventing and/or treating peripheral neuropathy; (e) preventing and/or treating pain; and/or (f) preventing and/or treating chronic constriction injury of neuropathic pain.

17. A use of the host cell according to claim 6, wherein it is used to prepare a composition, which is used for one or more uses selected from the following groups: (a) inhibiting TRPM8; (b) preventing and/or treating a TRPM8-related disease; (c) preventing and/or treating cold allodynia; (d) preventing and/or treating peripheral neuropathy; (e) preventing and/or treating pain; and/or (f) preventing and/or treating chronic constriction injury of neuropathic pain.

18. A use of the composition according to claim 7, wherein it is used to prepare a composition, which is used for one or more uses selected from the following groups: (a) inhibiting TRPM8; (b) preventing and/or treating a TRPM8-related disease; (c) preventing and/or treating cold allodynia; (d) preventing and/or treating peripheral neuropathy; (e) preventing and/or treating pain; and/or (f) preventing and/or treating chronic constriction injury of neuropathic pain.

19. The use according to claim 8, wherein the TRPM8-related disease is selected from the following groups: peripheral neuropathy, cold allodynia, pruritus, chronic constriction injury of neuropathic pain, or a combination thereof; and/or
the pain is selected from the following groups: chronic pain, cold allodynia pain, neuropathic pain of diabetic neuropathy, postoperative pain, osteoarthritis pain, rheumatoid arthritis pain, cancer pain, neuralgia, neuralgia Nociceptive pain, migraine, cluster headache, tension headache, fibromyalgia, neuropathic pain, static allodynia, cold allodynia, or combinations thereof.

20. A method of (a) inhibiting TRPM8; (b) preventing and/or treating TRPM8-related diseases; (c) preventing and/or treating cold allodynia; (d) preventing and/or treating peripheral neuropathy; (e) preventing and/or treating pain; and/or (f) preventing and/or treating chronic constriction injury of neuropathic pain, wherein the method includes the step of: administering the polypeptide or a pharmaceutically acceptable salt thereof according to claim 1 to a subject in need.

21. A method of (a) inhibiting TRPM8; (b) preventing and/or treating TRPM8-related diseases; (c) preventing and/or treating cold allodynia; (d) preventing and/or treating peripheral neuropathy; (e) preventing and/or treating pain; and/or (f) preventing and/or treating chronic constriction injury of neuropathic pain, wherein the method includes the step of: administering the fusion protein according to claim 3 to a subject in need.

22. A method of (a) inhibiting TRPM8; (b) preventing and/or treating TRPM8-related diseases; (c) preventing and/or treating cold allodynia; (d) preventing and/or treating peripheral neuropathy; (e) preventing and/or treating pain; and/or (f) preventing and/or treating chronic constriction injury of neuropathic pain, wherein the method includes the step of: administering the polynucleotide according to claim 4 to a subject in need.

23. A method of (a) inhibiting TRPM8; (b) preventing and/or treating TRPM8-related diseases; (c) preventing and/or treating cold allodynia; (d) preventing and/or treating peripheral neuropathy; (e) preventing and/or treating pain; and/or (f) preventing and/or treating chronic constriction injury of neuropathic pain, wherein the method includes the step of: administering the vector according to claim 5 to a subject in need.

24. A method of (a) inhibiting TRPM8; (b) preventing and/or treating TRPM8-related diseases; (c) preventing and/or treating cold allodynia; (d) preventing and/or treating peripheral neuropathy; (e) preventing and/or treating pain; and/or (f) preventing and/or treating chronic constriction injury of neuropathic pain, wherein the method includes the step of: administering the composition of claim 7 to a subject in need.


SEQUENCE LISTING (19 sequences)

SEQ ID NO: 1 - protein, 13 aa, synthetic construct
CRRDRARHYRQRC

SEQ ID NO: 2 - protein, 13 aa, synthetic construct
CRNSRAAHDSQKC

SEQ ID NO: 3 - protein, 13 aa, synthetic construct
CDHRRRQARYRRC

SEQ ID NO: 4 - protein, 13 aa, synthetic construct
CARDRARHYRQRC

SEQ ID NO: 5 - protein, 13 aa, synthetic construct
CRADRARHYRQRC

SEQ ID NO: 6 - protein, 13 aa, unidentified
CRRARARHYRQRC

SEQ ID NO: 7 - protein, 13 aa, synthetic construct
CRRDAARHYRQRC

SEQ ID NO: 8 - protein, 13 aa, synthetic construct
CRRDRGRHYRQRC

SEQ ID NO: 9 - protein, 13 aa, synthetic construct
CRRDRAAHYRQRC

SEQ ID NO: 10 - protein, 13 aa, synthetic construct
CRRDRARAYRQRC

SEQ ID NO: 11 - protein, 13 aa, synthetic construct
CRRDRARHARQRC

SEQ ID NO: 12 - protein, 13 aa, synthetic construct
CRRDRARHYAQRC

SEQ ID NO: 13 - protein, 13 aa, synthetic construct
CRRDRARHYRARC

SEQ ID NO: 14 - protein, 13 aa, synthetic construct
CRRDRARHYRQAC

SEQ ID NO: 15 - protein, 13 aa, synthetic construct
CRNDRARHYRQRC

SEQ ID NO: 16 - protein, 13 aa, synthetic construct
CRRSRARHYRQRC

SEQ ID NO: 17 - protein, 13 aa, synthetic construct
CRRDRARHDRQRC

SEQ ID NO: 18 - protein, 13 aa, synthetic construct
CRRDRARHYSQRC

SEQ ID NO: 19 - protein, 13 aa, synthetic construct
CRRDRARHYRQKC